(19) United States
(12) Patent Application Publication
(10) Pub. No.: US 2015/0291517 A1
(43) Pub. Date: Oct. 15, 2015

(54) BENZENESULFONAMIDE COMPOUNDS FOR SOMATIC EMBRYOGENESIS IN PLANTS

(71) Applicants: Martijn Fiers, Bennekom (NL); Kimberly Boutilier, De Meern (NL)

(72) Inventors: Martijn Fiers, Bennekom (NL); Kimberly Boutilier, De Meern (NL)

(21) Appl. No.: 14/647,717

(22) PCT Filed: Nov. 28, 2012
(86) PCT No.: PCT/IB2012/056790

§ 371 Date: May 27, 2015

(51) Int. Cl. C07C 311/21; A01H 4/00

(52) U.S. Cl. CPC C07C 311/21; A01H 4/005

(57) ABSTRACT

Benzenesulfonamide compounds potentiate 2,4-D induced embryogenesis in plants. In particular, 4-chloro-N-methyl-N-(2-methylphenyl)benzenesulfonamide and analogs induce somatic embryogenesis in plants. Methods of inducing somatic embryogenesis comprise exposing selected plant tissues, e.g. seed embryos, to auxins, e.g. 2.4-D and the benzenesulfonamide compounds. Compounds can be prepared by reacting sulfonyl chloride, an amine and pyridine in CH₂CI₂. Crude product is suspended in ethyl acetate and washed in sodium and potassium hydrogen sulphates and brine, then dried and filtered.

This invention relates to somatic embryogenesis (SE) in plants, which is the formation of plant embryos from vegetative or somatic plant cells, including somatic embryos made from seed embryos. The invention concerns compounds and composition which affect plant cells ability to form somatic embryos.

Plant regeneration and clonal propagation are important techniques in agricultural and horticultural sectors, where they are used to facilitate the breeding process, to propagate parental lines for hybrid seed production, and to propagate highly heterozygous or open pollinated varieties that are sold as plantlets.

One technique used to clonally propagate plant material is SE in which embryo-like structures can develop into fertile plants in a way analogous to zygotic embryos that develop in the seed.

SE has several advantages compared to other in vitro clonal propagation systems, such as the possibility to obtain a high yield of plants in a short time, the possibility to scale-up in liquid suspension cultures and synthetic seed technologies.

In the model system of Arabidopsis thaliana, immature zygotic embryos can be induced in a highly efficient manner to form somatic embryos with the aid of the synthetic auxin 2,4 dichlorophenoxyacetic acid (2,4-D). This is in contrast to zygotic embryos from dry seed, which have a reduced competence to form somatic embryos.

Considering plants generally, a complicating factor is a large variability in SE efficiency between different cultivars and species. Transferring SE protocols from model genotypes to other genotypes and plant species poses problems and germplasm recalcitrance of commercial cultivars is a major continuing problem. In order to be able to induce SE in recalcitrant cultivars an empirical approach, based on existing tissue culture protocols, hitherto, needs to be used to identify optimal conditions and inducer treatments for each individual genotype. These processes though are hugely time consuming and may not result in an efficient somatic embryogenesis protocol.

A genetic modification (GMO) approach, in which regeneration-promoting genes are (conditionally) over-expressed may be used, but this approach requires a transformable genotype.

The scientific literature comprises many publications of the various methods of somatic embryogenesis, each method involving culturing explants of tissue, cells or callus in a specifically elucidated growth medium comprising plant growth regulators (PGRs). Gaj M. D. (2004) et al. Plant Growth Regulation 43: 27-47 is a review article which summarises the general characteristics of SE and considers the various factors which are crucial for somatic embryo induction; including the growth medium compositions and plant growth regulators (PGRs). Of the PGRs used in somatic embryo induction media, 2,4-D is the more frequently reported example which induces an embryogenic response, whether alone or in combination with other PGRs, across a wide range of in vitro systems and plant tissues. Whilst 2,4-D is almost routinely reported as being used to induce SE, other auxins and PGRs, whether alone or in combination in growth media, have been reported to induce embryogenesis.

The published patent literature in the field of somatic embryogenesis to date is characteristically reflective of the science, whereby a method of embryogenesis is developed empirically for a particular plant species or cultivar, usually of an ornamental, horticultural or agricultural plant; but also including conifer trees.

Thus there is a need in the context of plant breeding and plant improvement to enhance or develop generically applicable methods for efficiently enhancing SE in a cost-effective manner.

The inventors have discovered that 4-chloro-N-methyl-N-(2-methylphenyl)benzenesulfonamide enhances the formation of somatic embryos from germinated seeds. The inventors have found that 4-chloro-N-methyl-N-(2-methylphenyl)benzenesulfonamide and similar compounds are excellent potentiators of 2,4-D induced embryogenesis in Arabidopsis. In particular, the inventors have discovered certain of these compounds induce SE in Arabidopsis in the presence of 2,4-D, some strongly so.

In accordance with a first aspect of the present invention, there is provided a compound or a salt or solvate of a compound of the formula

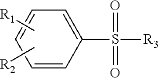

wherein either:
   R₁ and R₂ together form an aromatic ring that is fused to the phenyl ring to which R₁ and R₂ are attached, or
   R₁ and R₂ are independently selected from H, halogen, halohydrocarbyl, ether, nitro and C₁ to C₅ alkyl functional groups;
R₃ is NR₄R₅,
   (i) where R₄ is H or a C₁ to C₅ hydrocarbyl, and where R₅ is H, an amide or —C₆H₂R₆R₇R₈, where R₆, R₇ and R₈ are independently selected from at least one of H, C₁-C₆ hydrocarbyl, halogen, halohydrocarbyl, hydroxyl and an ether functional group, or
   (ii) where N, R₄ and R₅ together form a heterocyclic ring,
   wherein the compound is not one of the following:

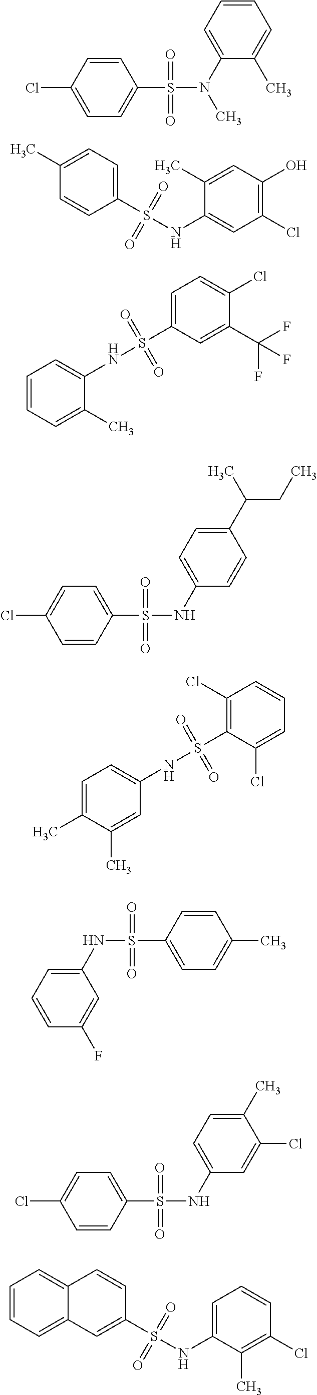

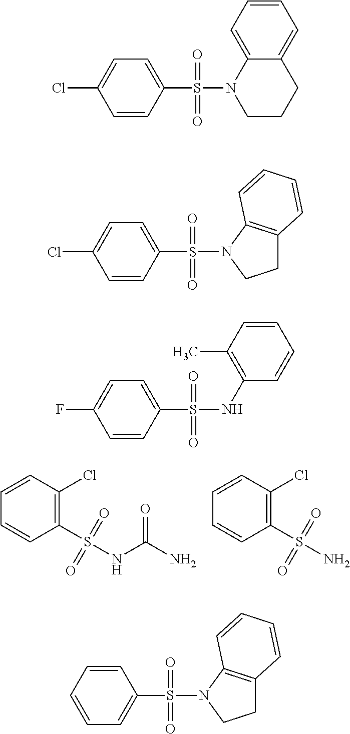

The compound preferably has the formula:

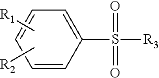

wherein:
R₁ and R₂ together form an aromatic ring that is fused to the phenyl ring to which R₁ and R₂ are attached, or R₁ and R₂ are independently selected from H, halogen, ether, nitro and C₁ to C₅ alkyl functional groups;
R₃ is NR₄R₅, (i) where R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring.

Preferably, neither R₄ nor R₅ is H.

In one embodiment, (i) R₄ is a C₁ to C₅ hydrocarbyl, and R₅ is —C₆H₂R₆R₇R₈, where R₆, R₇ and R₈ are independently selected from at least one of H, C₁-C₆ hydrocarbyl and an ether functional group, or (ii) N, R₄ and R₅ together form a heterocyclic ring. Preferably, R₄ is methyl or ethyl.

In an alternative embodiment, the compound is

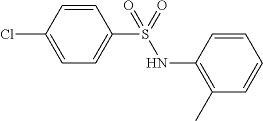 [SMILES: Cc1ccccc1NS(=O)(=O)c1ccc(Cl)cc1]

Other examples of the compound include:

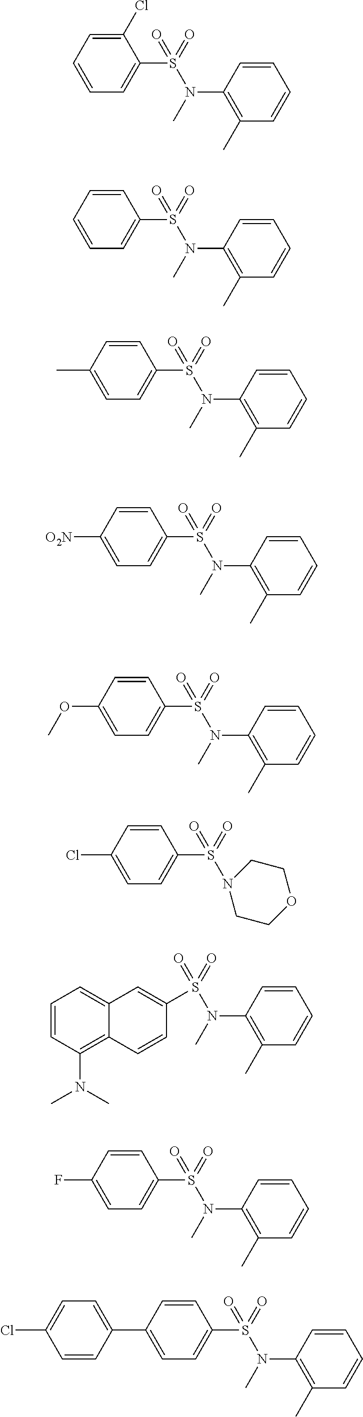

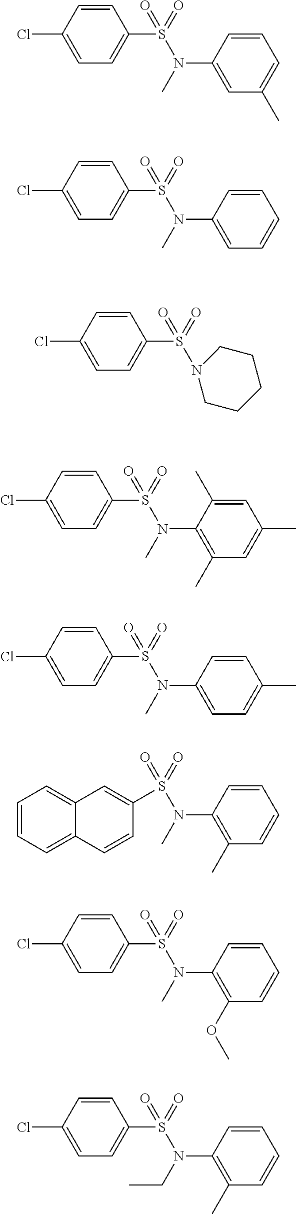

The compound may be synthesised using techniques that are well-known in the art. Necessary starting materials may be obtained by standard procedures of organic chemistry. Such a method is described herein.

In accordance with a second aspect of the present invention there is also provided a method of potentiating somatic embryogenesis or organogenesis in a plant, comprising exposing plant cells, plant tissue, plant parts or plant embryos to an auxin and to one or more compounds or salts or solvates of the compounds of the formula:

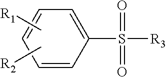

wherein:
either:
R₁ and R₂ together form an aromatic ring that is fused to the phenyl ring to which R₁ and R₂ are attached, or
R₁ and R₂ are independently selected from H, halogen, halohydrocarbyl, ether, nitro and C₁ to C₅ alkyl functional groups,

### R₃ is NR₄R₅,

(i) where R₄ is H or a C₁ to C₅ hydrocarbyl, and where R₅ is H, an amide or —C₆H₂R₆R₇R₈, where R₆, R₇ and R₈ are independently selected from at least one of H, C₁-C₆ hydrocarbyl, halogen, halohydrocarbyl, hydroxyl and an ether functional group, or
(ii) where N, R₄ and R₅ together form a heterocyclic ring,
   and then culturing the cells, tissue, part or embryo.

Protocols and techniques for culturing plant cells, tissues, parts or embryos will be well known to a person of average skill in the art for a wide range of plant species; see for example, the textbook “Plant Cell Culture” (2010) Michael R Davey and Paul Anthony (Wiley-Blackwell). Also, for example, the laboratory methods book “Plant Cell Culture Protocols” (2006) 2^{nd} edition, Victor M Loyola-Vargas and Felipe Vazquez-Flota (Humana Press).

Potentiating the SE activity of auxin is defined as increasing or decreasing the somatic embryo-inducing activity of auxin when used alone without the one or more compounds of the invention. Compounds in accordance with the invention which decrease the SE inducing activity of auxin are equally useful as compounds which increase SE activity as they may be used in studies to elucidate genetic and biochemical basis of SE in plants and to prevent over-proliferation of tissues in culture so that differentiation and subsequent plantlet growth may occur.

Advantageously, the ability of the present invention using certain of the defined compounds to chemically induce SE eliminates the need to create and market transgenic plants, allowing rapid and cost-effective innovation.

Advantageously, the methods of the invention rapidly induce/enhance SE in an inexpensive, non-GMO manner.

Also advantageously, in relation to industrial application, compounds for use in accordance with the invention can be directly tested and implemented in any SE protocol, including existing protocols, without having to perform additional studies to define specificity or mode of action of the compounds.

Advantageously, the invention offers plant breeders the capability of inducing or enhancing SE in a range of different crops.

The compounds of the invention may also be used in potentiating (i.e. increasing or decreasing) different types of auxin-mediated organogenesis, preferably to enhance plantlet formation.

In this specification, the term “aromatic ring” refers to both monocyclic and polycyclic aromatic rings. As discussed above, R₁ and R₂ together form an aromatic ring that is fused to the phenyl ring to which R₁ and R₂ are attached. The aromatic ring may be a 5 or 6-membered ring. The ring is preferably a hydrocarbyl ring, preferably a C₅ or C₆ hydrocarbyl ring. In one embodiment, R₁ and R₂ together form a phenyl ring that is fused to the phenyl ring to which R₁ and R₂ are attached to provide a naphthalene substituent. The aromatic ring may optionally be substituted, for example, with a C₁ to C₆ alkyl group, such as a methyl or ethyl group. Other possible substituents include ether and amines, such as OCH₃, OC₂H₅ and N(CH₃)₂.

The term “hydrocarbyl” refers to substituents consisting of hydrogen and carbon. Such groups may be saturated or unsaturated. For example, the hydrocarbyl may include one or more double or triple carbon-carbon bonds. The hydrocarbyl may be aliphatic or aromatic. Suitable hydrocarbyl groups include straight chain, branched chain and cyclic (e.g. alicyclic or aromatic) groups. The hydrocarbyl group may be substituted with, for example, a heteroatom such as O or S or a halogen atom. The term “halohydrocarbyl” refers to a hydrocarbyl that has been substituted with at least one halogen group. Suitable halogen groups include F, Cl, Br and I. Examples of halohydrocarbyl groups include fluoro-, chloro-, bromo- and iodo-hydrocarbyls. Suitable halohydrocarbyls include haloalkyls, such as halo(C₁-C₆) alkyls. Specific examples include mono-, di- or tri-substituted haloalkyls, such as trifluoromethyl. In one embodiment, at least one of R₆, R₇ and R₈ is a halohydrocarbyl, preferably a haloalkyl, such as a halo(C₁-C₆) alkyl, for example, trifluoromethyl. Other examples of halohydrocarbyls include halo-substituted aryls, such as halophenyl. In one embodiment, R₁ and R₂ are independently selected a halohydrocarbyl, such as a haloaryl, for example, a halophenyl.

Unless indicated otherwise, the term “alkyl” includes cyclic, straight and branched chain alkyl groups. References to individual alkyl groups such as “n-propyl” are specific for the straight chain version only and references to individual branched chain alkyl groups such as “isopropyl” are specific for the branched chain version only. For example, C₁ to C₄ alkyl includes methyl, ethyl, n-propyl, i-propyl, n-butyl, t-butyl and sec-butyl.

The term “heterocyclic” means a cyclic hydrocarbyl in which one of the carbon atoms in the ring has been substituted with a heteroatom. Examples of heteroatoms include nitrogen, phosphorus, sulphur and oxygen. Nitrogen and oxygen are preferred. The heterocyclic groups may be saturated, unsaturated, aliphatic and/or aromatic. The heterocyclic group is preferably unsaturated, although this unsaturated heterocyclic group may be substituted or fused with an aromatic substituent, such as an aryl (e.g. phenyl) ring. Suitable heterocyclic groups include monocyclic, fused, bridged, or spiro bicyclic heterocyclic ring system(s). Monocyclic heterocyclic rings contain from about 3 to 12 (suitably from 3 to 7, preferably 5 or 6) ring atoms, with from 1 to 5 (suitably 1, 2 or 3) heteroatoms selected from nitrogen, oxygen or sulfur in the ring. Suitable unsaturated heterocyclic groups include piperidinyl, pyrrolidinyl and morpholinyl groups. Each of these may optionally be fused with a phenyl group.

Preferably, the compounds are of the formula:

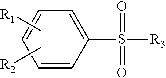

wherein:
R₁ and R₂ together form an aromatic ring that is fused to the phenyl ring to which R₁ and R₂ are attached, or R₁ and R₂ are independently selected from H, halogen, ether, nitro and C₁ to C₅ alkyl functional groups;
R₃ is NR₄R₅, (i) where R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring.

Where R₁ and/or R₂ are halogen, the halogen may be selected from F, Cl, Br and I, preferably, F, Cl and Br. In one embodiment, only one of R₁ and R₂ is halogen; the other is preferably hydrogen.

Where R₁ and/or R₂ is C₁ to C₅ alkyl, the alkyl group may be methyl, ethyl, propyl (e.g. n- and i-propyl), butyl (n-, sec-, i- and t-butyl) or pentyl (e.g. cyclopentyl). Preferred alkyl groups include methyl and ethyl. In one embodiment, only one of R₁ and R₂ is a C₁ to C₅ alkyl; the other is preferably hydrogen.

In a group of compounds, one or both of R₁ and R₂ is hydrogen.

Where R₁ and/or R₂ is an ether, the ether is selected from groups of the formula ORₐ, where Rₐ is an alkyl group, preferably a C₁ to C₆ alkyl group. Preferred alkyl groups include methyl and ethyl. Examples include—OCH₃ and —OC₂H₅. In one embodiment, only one of R₁ and R₂ is a ether; the other is preferably hydrogen.

In one embodiment, R₁ and/or R₂ is a nitro (—NO₂) group. Preferably, only one of R₁ and R₂ is a nitro group; the other is more preferably hydrogen.

In a group of preferred compounds, R₁ is hydrogen, and R₂ is a functional group other than hydrogen (i.e. halogen, halohydrocarbyl, ether, nitro and C₁ to C₅ alkyl) that is ortho, meta or para, preferably para to the sulfonyl group.

Where R₁ and R₂ together form an aromatic ring that is fused to the phenyl group to which R₁ and R₂ are attached, the aromatic ring is preferably a phenyl ring.

R₄ may be H or a C₁ to C₅ hydrocarbyl. Preferably, R₄ is H or a C₁ to C₅ alkyl, more preferably methyl or ethyl.

In one embodiment, R₄ is H or a C₁ to C₅ hydrocarbyl, and R₅ is H or —C₆H₂R₆R₇R₈, where R₆, R₇ and R₈ are independently selected from at least one of H, C₁-C₆ hydrocarbyl, halo, halohydrocarbyl and an ether functional group. In a preferred embodiment, R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group.

Where at least one of R₆, R₇ and R₈ is a C₁-C₅ alkyl, the C₁ to C₅ alkyl is preferably a methyl, ethyl group, propyl (n- or i-propyl) or butyl (n-, i-, s- or t-butyl) group. Methyl, ethyl and s-butyl are preferred. In one embodiment, only one of R₆, R₇ and R₈ is a C₁-C₅ alkyl, the remainder are preferably hydrogen. In another embodiment, all three of R₆, R₇ and R₈ is a C₁ to C₅ alkyl, preferably methyl. These alkyl groups may preferably be at the 2-, 4- and 6-positions of the —C₆H₂R₆R₇R₈ aromatic ring.

Where at least one of R₆, R₇ and R₈ is an ether group, the ether may be of the formula ORₐ where Rₐ is an alkyl group, preferably a C₁ to C₆ alkyl group. Preferred alkyl groups include methyl and ethyl. Preferably only one of R₆, R₇ and R₈ is an ether group (e.g. OCH₃). This ether group may be at the 2, 4 or 6 position, preferably the 2-position, of the —C₆H₂R₆R₇R₈ aromatic ring.

Where at least one of R₆, R₇ and R₈ is halogen, the halogen may be F, Cl, Br or I. Halogen groups as R₆, R₇ and R₈ are not generally preferred.

Where at least one of R₆, R₇ and R₈ is halohydrocarbyl, the halohydrocarbyl may be trifluoromethyl. Halohydrocarbyl groups as R₆, R₇ and R₈ are not generally preferred.

In a group of preferred compounds, one or two of R₆, R₇ and R₈ is hydrogen. Preferably, two of R₆, R₇ and R₈ is hydrogen.

Where R₆ is hydrogen and at least one of R₇ and R₈ is a non-hydrogen functional group, the non-hydrogen functional group may be at the ortho, meta or para position of the —C₆H₂R₆R₇R₈ aromatic ring.

In one embodiment, R₄ is H or a C₁ to C₅ hydrocarbyl, and R₅ is H or an amide. This embodiment is not preferred. However, where R₅ is amide, the amide may be of the formula:

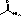

In another group of preferred compounds, R₃ is NR₄R₅, where N, R₄ and R₅ together form a heterocyclic ring, preferably a 6-membered heterocyclic ring.

N, R₄ and R₅ may together form a piperidinyl, pyrrolidinyl and morpholinyl ring.

In another group of preferred compounds, a further aromatic ring, for example, a phenyl ring is fused to said heterocyclic ring.

Preferred examples of R₃ are selected from:

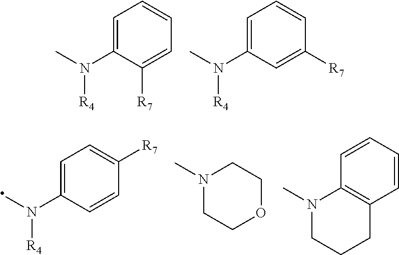

where R₄ is H, CH₃ or C₂H₅; and R₇ is selected from methyl, ethyl, propyl, butyl and OCH₃, or wherein R₃ is

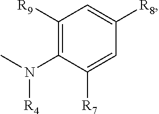

where R₄ is H, CH₃ or C₂H₅; and R₇, R₈ and R₉ are each methyl.

Preferably,

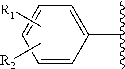

is selected from:

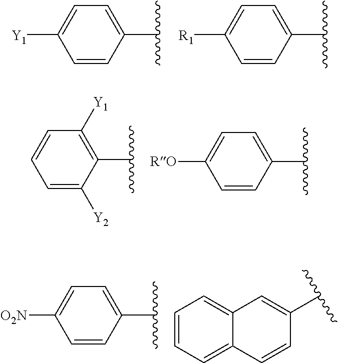

where Y₁ and Y₂ are independently selected from F, Cl, Br, I,
R₁ is methyl or ethyl, and R″ is methyl or ethyl.

For the avoidance of doubt, the preferred definitions of R₁, R₂, R₄, R₅, R₆, R₇ and R₈ may be applied to all aspects of the present invention e.g. both the first and second aspect of the invention.

Specific examples of compounds according to the invention are shown in FIGS. 1 and 4 of the drawings.

For the avoidance of doubt, the compounds of the present invention may be used in salt or solvated form

In accordance with the method of the invention, the plant cells, tissue, part or embryo may be, or may include, callus. Also, in accordance with the method of the invention, an embryo used as starting material may itself be a somatic embryo. Often a somatic embryo may be used to generate more somatic embryos in a process known as secondary somatic embryogenesis and the method of the invention defined herein includes secondary somatic embryogenesis.

In certain embodiments the plant cells, tissue, part or embryo may be exposed to the auxin and the one or more compounds substantially simultaneously.

In other embodiments the plant cells, tissue, part or embryo may be exposed to the auxin followed by the one or more compounds whether separately, sequentially or simultaneously.

In yet further embodiments, the plant cells, tissue, part or embryo may be exposed to the one or more compounds, whether separately, sequentially or simultaneously, followed by the auxin.

In other embodiments of the invention, there may be a first period of exposure followed by a second period of culturing in the absence of either the auxin and/or one or more of the compounds.

In any of the embodiments of the method of the invention, the exposing of the plant cells, tissue, part or embryo may take place in a liquid medium. The exposure may also take place at some stage in liquid medium, semi-solid medium or via solid medium, or use of all three in any desired order at any desired time during the conduct of the method of the invention.

Further, the culturing of cells, tissue, part or embryo following any exposure may take place on a solid medium.

In methods of the invention, an optimal window of culture time for the plant cells, tissue, part or embryo (including seeds/seedlings) with the auxin and compound of the invention may be determined in a routine way by a person of skill in the art. In preferred methods, the auxin and compound of the invention (whether used separately, sequentially or simultaneously) are applied within the first three days or culture, more preferably the first two days or first day. In other embodiments, the exposure to compound of the invention (with auxin whether applied separately, sequentially or simultaneously) may be no longer than about two days, preferably no longer than about three days from the start of the culturing process. In preferred embodiments of generating somatic embryos from germinating seeds, the culture time with auxin and compound of the invention no more than about a day, preferably no more than about two days.

The auxin used in accordance with the invention may be selected from one or more of: indole-3-acetic acid (IAA), indole-3-butyric acid (IBA), 4-chloroindole-3-acetic acid (4-Cl-IAA), 2-phenylacetic acid (PAA), 2.4-dichlorophenoxyacetic acid (2,4-D), α-napthalene acetic acid (α-NAA), 2-methoxy-3,6-dichlorobenzoic acid, 4-amino-3,5,6-trichloropicolinic acid. Any auxin, whether natural or synthetic may be used.

In preferred methods according to the invention, the auxin is 2,4 dichlorophenoxyacetic acid (2,4-D).

The invention also provides a method of generating plantlet or plant, comprising producing a somatic embryo as described herein and then regenerating the plantlet or plant from the embryo.

The invention additionally provides a composition for potentiating somatic embryogenesis or organogenesis in plants comprising an auxin and one or more compounds as hereinbefore described. The invention further includes compounds as hereinbefore described for use in potentiating somatic embryogenesis or organogenesis in a plant cell, tissue, part or embryo.

The invention also further includes compounds as hereinbefore described for use simultaneously, sequentially or separately with an auxin in the potentiation of somatic embryogenesis or organogenesis in a plant cell, tissue, part or embryo.

Also provided in accordance with the invention is a solid or liquid plant culture medium comprising one or more compounds as hereinbefore described; optionally further comprising an auxin.

The invention also includes a kit for potentiating somatic embryogenesis or organogenesis in plants, comprising a first container containing a substance which is or comprises one or more compounds as hereinbefore described, and a second container containing a substance which is or comprises an auxin.

In accordance with the invention, the one or more compounds are used at an appropriate concentration, preferably to enhance auxin-mediated SE when they are used at a concentration in the nanomolar to micromolar to millimolar range, preferably in the nanomolar to micromolar range. In particularly preferred embodiments the compounds are used in the range 0.1 to 100 micromolar, more preferably 1 to 50 micromolar.

Preferably, the one or more compounds used in the method of the invention increase the level of SE compared to the auxin alone. A comparative experiment and therefore comparative measurement is preferably made. A preferred measurement of level of SE is the number or weight of embryos produced from a plant cell, tissue, part, embryo, seedling or callus. The number or weight of embryos may be expressed in relation to the experimental material of cells, tissues, parts, embryos, seedlings or callus, i.e. a preferred measure is percentage of seedlings with embryo(s). In preferred embodiments seeking increased level of SE compared to auxin alone, the level of SE may be expressed as a ratio of embryos as measured per unit of experimental material, i.e. number or weight of embryos with one or more compounds of the invention and auxin:number or weight of embryos with auxin alone.

Somatic embryos are readily recognised by a person of average skill in the art. There are distinct morphological characteristics, for example a more mature somatic embryo is bipolar, usually lacking trichomes, is not connected to the underlying vascular tissue and is easily removed from the explant. Younger somatic embryos may be globular in shape, and as above do not contain trichomes and are not connected to the underlying vascular tissue of the explant. Also, there are gene expression markers measurable by qPCR. Such markers show an at least two-fold increase in plant tissue treated with an auxin plus compounds of the invention compared to a control plant tissue treated with just the auxin.

Markers for determining SE may include one or more of: At1g48130 1-cysteine peroxiredoxin 1; At2g34870 Hydroxyproline-rich glycoprotein family protein (MEE26); At4g28520 CRUCIFERIN 3; At5g44120 CRUCIFERIN 1; At1g73190 Aquaporin-like superfamily protein (TIP3); At3g22640 Cupin family protein (PAP85); At3g53040 Putative ate embryogenesis abundant protein; At4g25140 OLEOSIN 1; At3g22490 Seed maturation protein; At2g18340 Late embryogenesis abundant domain-containing protein; At2g41280 LEA protein M10; At5g01300 PEBP (phosphatidylethanolamine-binding) family protein; At5g40420 OLEOSIN 2; At4g27140 Seed storage albumin 1; At2g41260 Glycine-rich protein/LEA (M17); At3g01570 OLEOSIN family protein; At3g22060 Receptor-like protein kinase-related family protein; At1g05510 Protein of unknown function (DUF1264); At5g52300LTR 65/desiccation-responsive protein 29B; At2g42540 Cold-regulated 15a; At5g52310 LTR 78/desiccation-responsive protein 29A; At3g50970 Dehydrin family protein; At2g47770 TSPO protein-related; At2g44130 Galactose oxidase/F-box//kelch repeat superfamily protein; At3g13310 Chaperone DnaJ-domain superfamily protein; At3g59930 Encodes a defensin-like (DEFL) family protein; At5g48540 Receptor-like protein kinase-related family protein; At5g64120 Peroxidase superfamily protein; At5g01740 Nuclear transport factor 2 (NTF2) family protein; At1 g64370 Unknown protein; At3g58450 Adenine nucleotide hydrolases-like superfamily protein; At1g47980 Unknown protein; At3g28740 Cytochrome P450 superfamily protein; At5g42290 transcription activator-related; At3g03620 MATE efflux family protein; At4g36040 Chaperone DnaJ-domain superfamily protein; At1g17830 Protein of unknown function (DUF789); At2g19900 NADP-malic enzyme 1; At4g39130 Dehydrin family protein; At1g21680 DPP6 N-terminal domain-like protein; At1g77120 Alcohol dehydrogenase 1; At2g23640 Reticulan like protein B13; At3g54940 Papain family cysteine protease; At4g38620 Myb domain protein 4; At1g23070 Protein of unknown function (DUF300); At2g37770 NAD(P)-linked oxidoreductase superfamily protein; At2g02930 Glutathione S-transferase F3; At2g15490 UDP-glycosyltransferase 73B4 At2g38905 Low temperature and salt responsive protein family.

Alternatively, or in addition, the following one or more markers determinable by qPCR may be used to identify SE caused by an auxin and compounds of the invention when they are measured as being repressed or decreased in expression, by at least two-fold compared to a control plant tissue treated with the auxin alone: At5g38700 Unknown; At4g26880 Stigma-specific Stig1 family protein; At1g15580 AUXIN-INDUCIBLE 2-27; At4g02160 Unknown; At1g65310 XYLOGLUCAN ENDOTRANSGLUCOSYLASE/HYDROLAS; At1g33280 NAC DOMAIN CONTAINING PROTEIN 15; At1g07690 Unknown; At1g75250 RADIALIS-LIKE SANT/MYB 3; At4g31910 HXXXD-type acyl-transferase family protein; At4g04460 Saposin-like aspartyl protease family protein; At2g43140 Basic helix-loop-helix (bHLH) DNA-binding superfamily protein; At1g29090 Cysteine proteinases superfamily protein; At3g61930 Unknown; At2g32610 CELLULOSE SYNTHASE LIKE B1; At4g28680 Stress-induced tyrosine decarboxylase (TyrDC); At2g03850 Late embryogenesis abundant protein (LEA) family protein.

The markers for determining SE may include one or more of the genes described in publicly available embryo transcriptome data sets, including, but not limited to those described at Genevestigator (https://www.genevestigator.com/gv/) or The Bio-Array Resource for Plant Biology (http://bar.utoronto.ca/welcome.htm). Specific examples of such genes include LEAFY COTYLEDON1 (LEC1), LEAFY COTYLEDON2 (LEC2), FUSCA3 (FUS3) and WUSCHEL-related homeobox 2 (WOX2).

In all aspects, the invention is applicable to a wide range of plant species, including trees, crop plants, horticultural varieties and ornamentals; including cycads, conifers, angiosperm monocots or dicots Without purporting to be a comprehensive or exhaustive list of plants susceptible to the compounds and methods of the invention, the following are a list of genera of such plants: e,g, Abies, Pinus, Picea, Tsuga, Pseudotsuga, Thuja, Juniperus, Larix, Taxus and Sequoia. Other plants include, but are not limited to, the genera Elaeis, Phoenix, Eucalyptus, Quercus, Vitis, Malus, Triticum, Oryza, Glycine, Avena, Brassica, Saccharum, Hordeum, Fagopyrum, Gossypium, Beta, Arachis, Humulus, lopomea, Musa, Manihot, Coffea, Camellia, Rosa, Coca, Canabis, Papaver, Carica, Cocos, Daucus, Medicago, Zea, Theobroma, Abies, Acer, Alnus, Arbutus, Asimina, Betula, Carpinus, Carya, Castanea, Celtis, Cercis, Chamaecyparis, Cornus, Cryptomeria, Eucalyptus, Fagus, Fraxinus, Gleditsia, Gymnocladus, Hamamelis, Juglans, Juniperus, Larix, Liriodendron, Magnolia, Malus, Morus, Nyssa, Ostrya, Picea, Pinus, Platanus, Populus, Prunus, Pseudotsuga, Pielea, Quercus, Rhamnus, Rhus, Salix, Sambucus, Sassafras, Sequoia, Solanum, Sorbus, Taxus, Thuja, Tilia, Tsuga, Ulmus, and Viburnum.

### BRIEF DESCRIPTION OF THE DRAWINGS

Embodiments of the invention are further described hereinafter with reference to the accompanying drawings, in which:

FIG. 1 shows the results of an experiment testing 25 μM 4-chloro-N-methyl-N-(2-methylphenyl)benzenesulfonamide (compound #3) and similar compounds and analogues at the same concentration for SE activity in the presence of 1 μM 2,4-D to show structure-activity relationships. Error=SD (n=4).

FIG. 2 shows: the structure of compound C#3 (A); dose response curve for somatic embryogenesis for compound C#3 (B); and enhancement by C#3 of the number of somatic embryo-producing seedlings in different Arabidopsis ecotypes (C).

FIG. 3 shows the results of experiments testing for structure-activity relationships for SE on Arabidopsis seedlings for a further range of analogue compounds (as shown in FIG. 4), tested at 25 μM of compound in combination with 1 μM2,4-D. The bar with 2,4-D represents the control with only 1 μM of 2,4-D. All experiments were performed in containers. Error=SD (n=4).

FIG. 4 shows the structures of the compounds for which SE-enhancing activity is shown in FIG. 3. The number (%) shows the percentage of somatic embryos formed.

FIG. 5 shows the results of dose-response experiments with C#3 in combination with 2,4-D. The effect of C#3 on SE was tested with an increasing concentration of C#3, ranging from none to 50 μM of C#3, in the presence of 1 μM 2,4-D. All experiments were performed in containers. Error=SD (n=4). Also shown are pictures allowing comparison of Arabidopsis seedlings with and without somatic embryos.

FIG. 6 shows a time course of the C#3 effect of enhancement of 2,4-D stimulated somatic embryogenesis in Arabidopsis seedlings.

FIG. 7 shows the results of an experiment in which increasing concentrations of 2,4-D are exposed to Arabidopsis seedlings in the presence or absence of C#3 (A).

FIG. 8 shows the effect of 25 μM C#3 on NAA induced somatic embryogenesis in Arabidopsis seedlings.

### EXAMPLE 1

### Screening of Compounds for Somatic Embryogenesis Activity

The chemical screen was based on a modified version of an Arabidopsis somatic embryogenesis protocol that uses germinated seeds as explants (see Kobayashi et al., 2010 Kobayashi T, Nagayama Y, Higashi K and Kobayashi M. (2010). Establishment of a tissue culture system for somatic embryogenesis from germinating embryos of Arabidopsis thaliana Plant Biotech. 27: 359-364). In the original protocol, seeds were germinated on solid medium for one day, then the embryos were removed from the seed coat and transferred to solid media containing 4.5 μM 2,4-D to stimulate somatic embryo development. In this example, the protocol is simplified by germinating and culturing the seeds continuously in liquid medium containing 1 μM 2,4-D. Under optimized culture conditions (circa 30 seeds in 30 ml medium in 100 ml containers) approximately 18% of Col0 seeds form somatic embryos.

The LATCA (Library of AcTive Compounds on Arabidopsis (LATCA) library (see Zhao et al., (2007) Zhao Y, Chow T F, Puckrin R S, Alfred S E, Korir A K, Larive C K, Cutler S R (2007) Chemical genetic interrogation of natural variation uncovers a molecule that is glycoactivated. Nat Chem Biol. 3:716-21. was screened for small molecules that enhance the frequency of somatic embryo induction from germinated seeds. The screens are performed in 96-well microtitre plates. For the primary screen, 2.5 μl of each compound (2.5 mM stock in dimethyl sulfoxide (DMSO)) was added to 250 μl of half strength Murashige and Skoog (MS) medium with micro and micro elements and vitamins (Duchefa) and 1% (w/v) sucrose (pH 5.8, MS10) containing 1 μM 2,4-dichlorophenoxyacetic acid (2,4-D) in 96-well microtitre plates.

Approximately 10 surface-sterilized Columbia (Col-0) seeds were added to each well. The seeds were stratified at 4° C. in the dark for two days and then grown in on a rotary shaker (100 rpm/min) on a 16 h/8 h day/night cycle at 25° C. The number of seedlings that formed somatic embryos was counted two weeks later. Candidate compounds identified in the first screen were reordered (Chembridge or Maybridge), and re-tested in a second screen under the same conditions as the primary screen, but with a larger sample number (one 96-well microtitre plate/compound). Positive compounds were analysed using ChemMine (http//bioweb.ucr.edu/ChemMineV2/), ChemSpider (http//www.chemsider.com/) and the ZINC database (http://zinc.docking.org) for analogues with a known biological function.

Analogues of compound 3 (C3, 4-chloro-N-methyl-N-(2-methylphenyl)benzenesulfonamide, ID 5601004) were ordered (Chembridge or Maybridge) or synthesized.

The following general protocol for the synthesis of benzenesulfonamides was used:

Sulfonyl chloride (1 mmol), the amine (2 mmol) and pyridine (1 mmol) were dissolved in CH₂Cl₂ (10 ml) and the resulting mixture was stirred overnight at room temperature. The reaction was checked by thin layer chromatography (TLC) and the solvent was evaporated. The crude compound was taken up in EtOAc (25 ml) and washed with 1N KHSO₄ (25 ml), 1N NaHCO₃ (25 ml) and brine (25 ml). The organic layer was dried over Na₂SO₄, filtered and concentrated in vacuo. The compound was purified using recrystallization from EtOAc/Hexanes. If further purification was necessary, the compound was purified using silica gel column chromatography. The process delivers yields in the range 51-73%.

The effect of the compounds on seedlings, without 2,4-D, was tested by germinating seedlings on MS10 solidified with 1% agar, and supplemented with 25 μM compound or DMSO. The ENOD4L promoter comprises a 1035 bp promoter from Brassica napus (AB098076.1), which was cloned in front of green fluorescent protein (GFP). A 3110 bp DNA fragment comprising 1292 bp upstream of the LEC1 translational start site and the entire LEC1 protein coding region was cloned in front of green fluorescent protein (GFP). These vectors were transformed to Arabidopsis ecotype Columbia (Col-0).

Using the lower response, higher throughput screen (10 seeds per well), twenty seven compounds were identified that associated with somatic embryo development. These compounds were re-tested in a 96-well plate (circa 1000 seeds per compound). The compound 4-chloro-N-methyl-N-(2-methylphenyl)benzenesulfonamide was chosen for further analysis.

All subsequent experiments were performed under the conditions described above, but using 100 ml containers (Greiner) containing 30 ml of medium media using approximately 30 seeds/container. All hormones were ordered from Duchefa and Dicamba from Sigma-Aldrich.

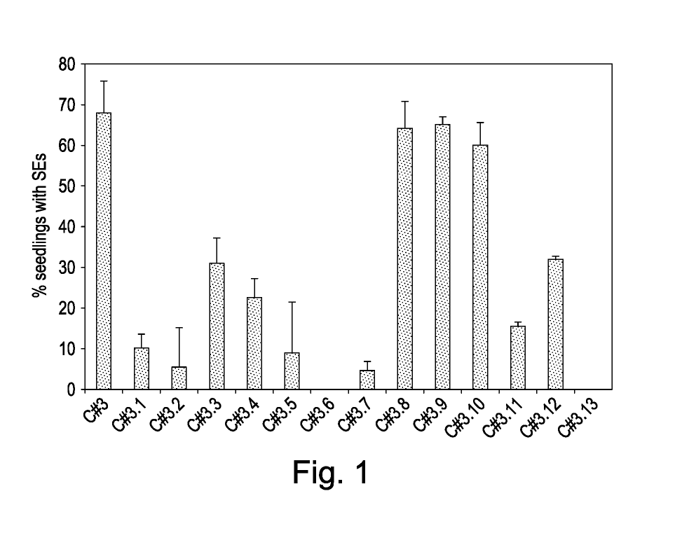

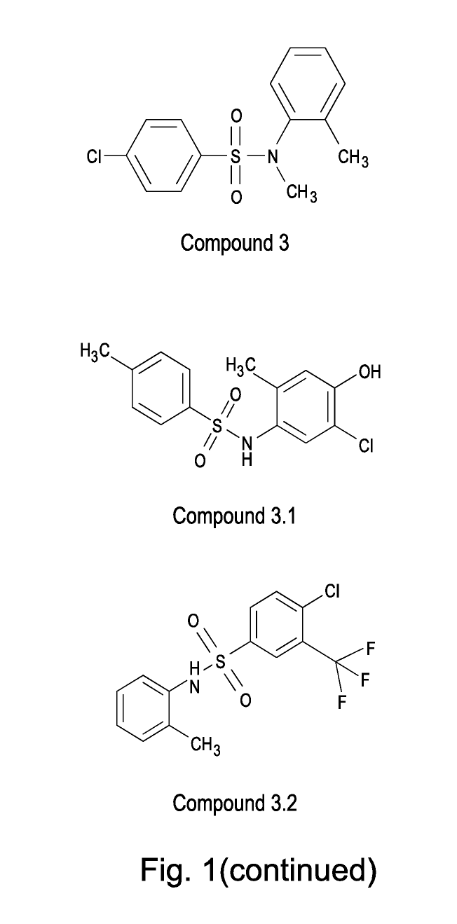

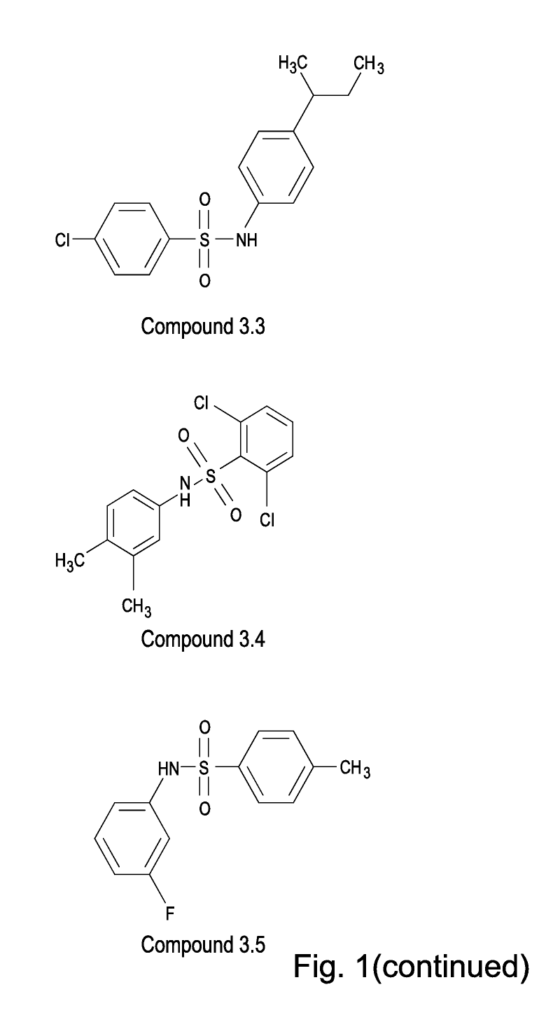

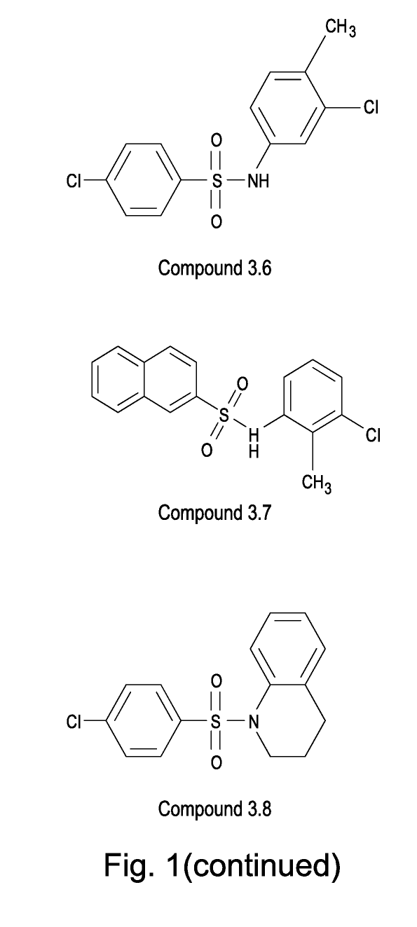

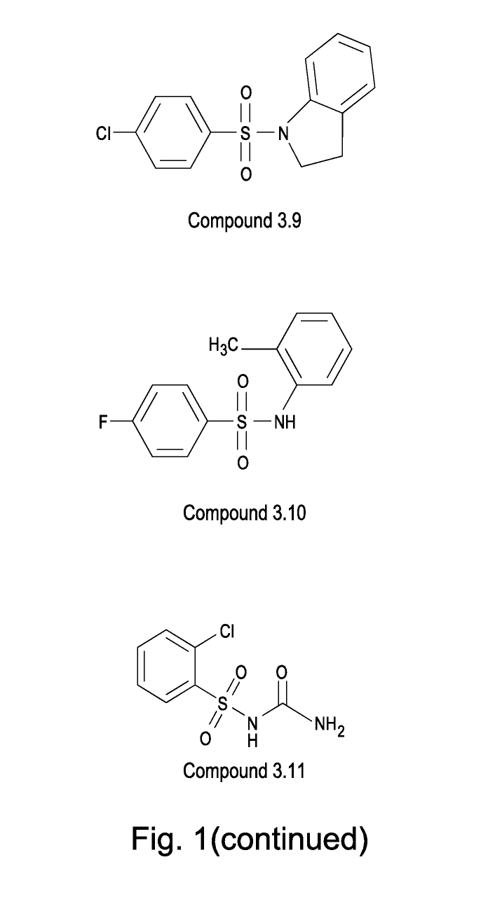

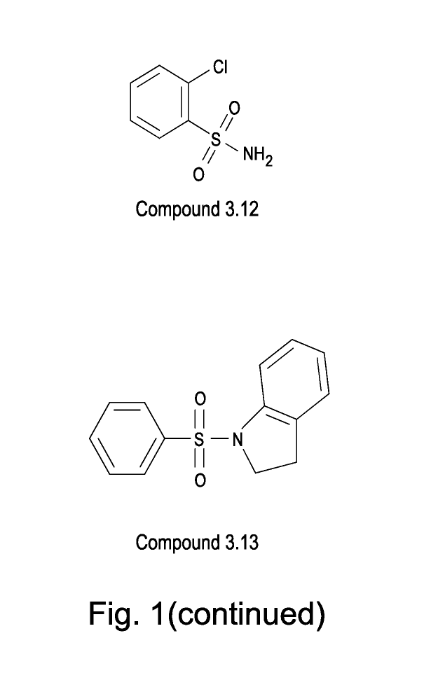

Referring to FIG. 1, this shows the results of testing compounds C#3-C#3.13 in the presence of 1 μM 2,4-D. Each of compounds #3, #3.3, #3.4, #3.8, #3.9, #3.10 and #3.12 significantly increases the level of SE in Arabidopsis seedlings, as measured by the percentage of seedlings with somatic embryos, compared to a control of 1 μM 2,4-D alone. Compounds #3, #3.8, #3.9 and #3.10 are particularly potent inducers.

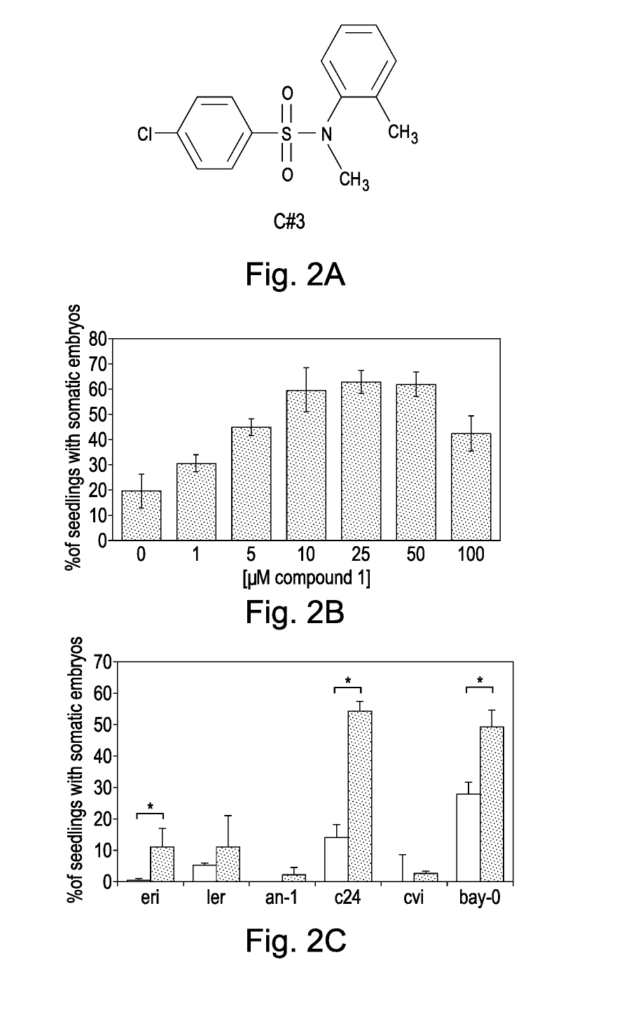

The compound C#3, 4-chloro-N-methyl-N-(2-methylphenyl)benzenesulfonamide (FIG. 2A), induced the highest percentage of seedlings with somatic embryos and was chosen for further analysis. A dose-response analysis was performed under optimized culture conditions (circa 30 seeds in 30 ml medium) and showed that C#3 is most effective between 10 and 50 μM, where it induced somatic embryo development in approximately 70% of seedlings, compared to circa 20% of the seedlings in the control (FIG. 2B). The ability of Cl to enhance somatic embryo production was not limited to Col0, as C#3 also enhanced the number of somatic embryo-producing seedlings in different Arabidopsis ecotypes (FIG. 1C). All subsequent experiments were performed using 25 μM C#3 in under these optimized tissue culture conditions.

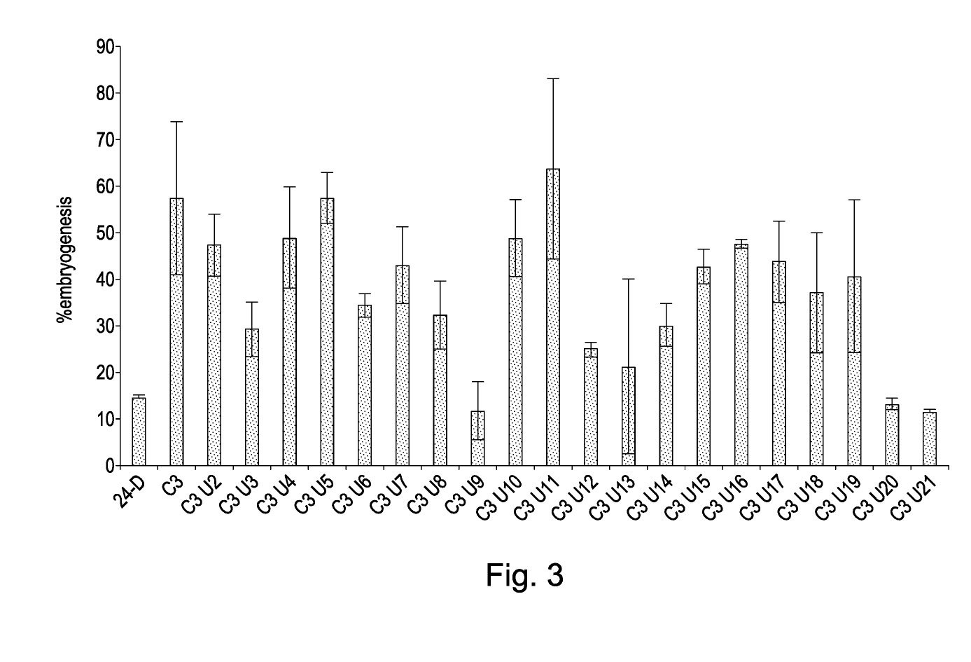

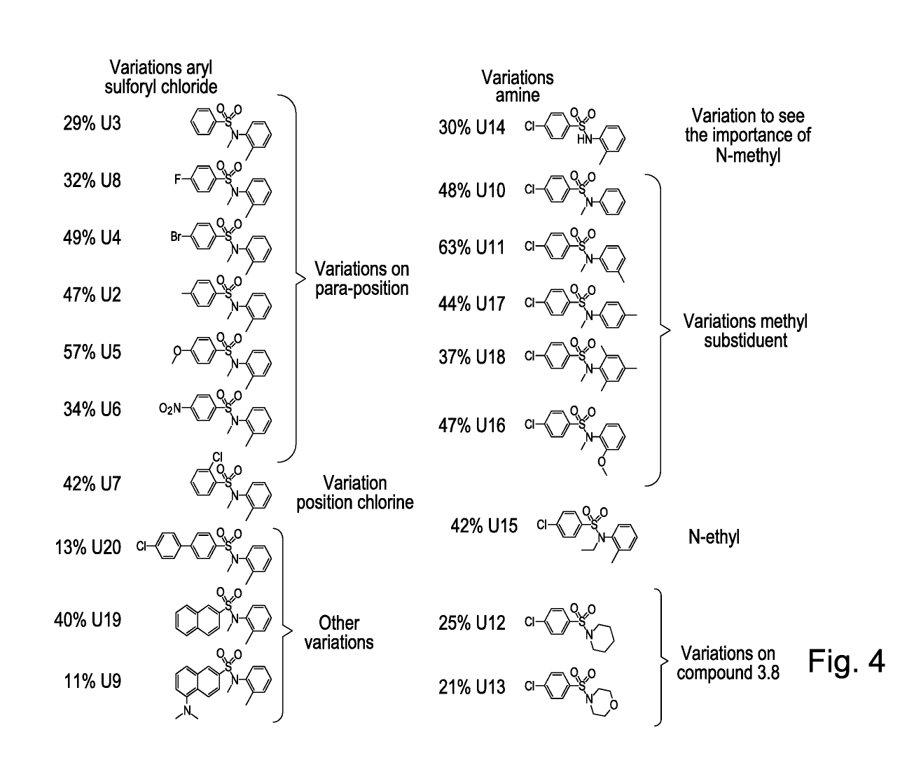

Referring to FIG. 3, this shows the results of screening a range of synthesised compounds denoted U2-U21 and as shown in FIG. 4.

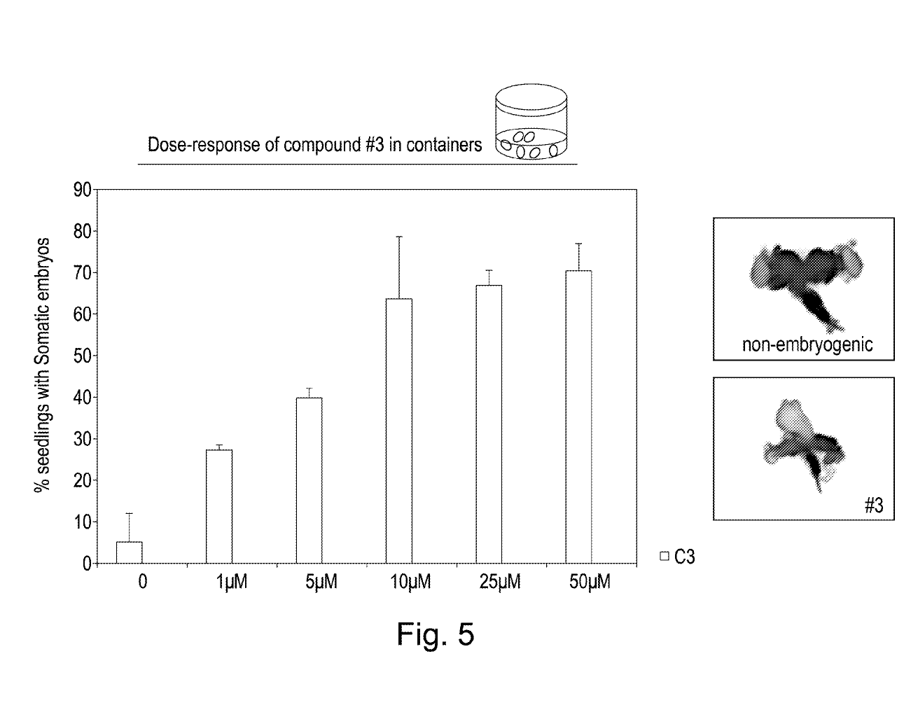

Referring to FIG. 5, this shows how increasing concentrations of compound 3 in combination with a fixed concentration of 2,4-D in containers of Arabidopsis seedlings increases the level of SE, as measured as a percentage of seedlings with somatic embryos.

### EXAMPLE 2

### Effect of 4-chloro-N-methyl-N-(2-methylphenyl)benzenesulfonamide (Compound C#3) on Development of Somatic Embryos

Control and C#3-treated cultures were examined at different time points in their development to determine the effect of C#3 on the course of somatic embryo development. Differences between the control and C#3-treated cultures were visible at about four days of culture. The epidermal and cortex cells of the root elongation zone and hypocotyl in the control seedlings increased in size, but not in the C#3-treated seedlings. On the sixth day of culture the differences between the two cultures was more pronounced. The majority of seedlings in the control cultures had a hypocotyl-root region, that was characterized by epidermal and cortical cell layers that had loosened or detached from the underlying tissue, Vascular-derived callus formed above the meristematic zone and in the hypocotyl. The C#3-treated seedlings also showed signs of cell loosening and detachment in the hypocotyl and root, but to a lesser extent than the control. The C#3-treated seedlings formed a large, connected mass of vascular-derived callus in the root/hypocotyl region. Callus was present on the tips of the cotyledons in circa 10% of the control seedlings, and up to 70% of the C#3 treated seedlings. This callus comprised loose, large cells on the outside, and smaller, denser cells on the on inside.

On the tenth day of culture, light green embryogenic tissue and embryo were visible at the shoot apex in both control and C#3-treated cultures, but the number of embryogenic seedlings was much higher in the C#3-treated cultures compared to control cultures.

The timing and location of somatic embryo formation in control and compound-treated cultures was examined in more detail using two early embryo expressed marker genes, ENOD4-like (ENOD4L::GFP), and LEAFY COTYLEDON1 (LEC1::LEC1:GFP). Expression of ENOD4L::GFP and LEC1:GFP in zygotic embryos was observed from the zygote- and two-celled embryo proper stage onward, respectively. Expression of both markers was first detected in the C#3-treated cultures starting from day four of culture, where GFP-positive sectors were observed in the region of the shoot meristem. The GFP-positive sectors increased in size over the next few days, and on the sixth day of culture, GFP-positive globular embryos could be distinguished. The only difference between the C#3-treated and control cultures with respect to embryo marker expression was the number of seedlings displaying GFP fluorescence, which was higher in the C#3-treated cultures. Without wishing to be bound by any particular theory, the observation that somatic embryo induction follows the same origin and timing in control and C#3-treated cultures, suggests that C#3 enhances developmental processes that are already initiated by 2,4-D, rather than by activation of novel signalling pathways.

### EXAMPLE 3

### Developmental Competence of Seeds for 4-chloro-N-methyl-N-(2-methylphenyl)benzenesulfonamide (Compound C#3)

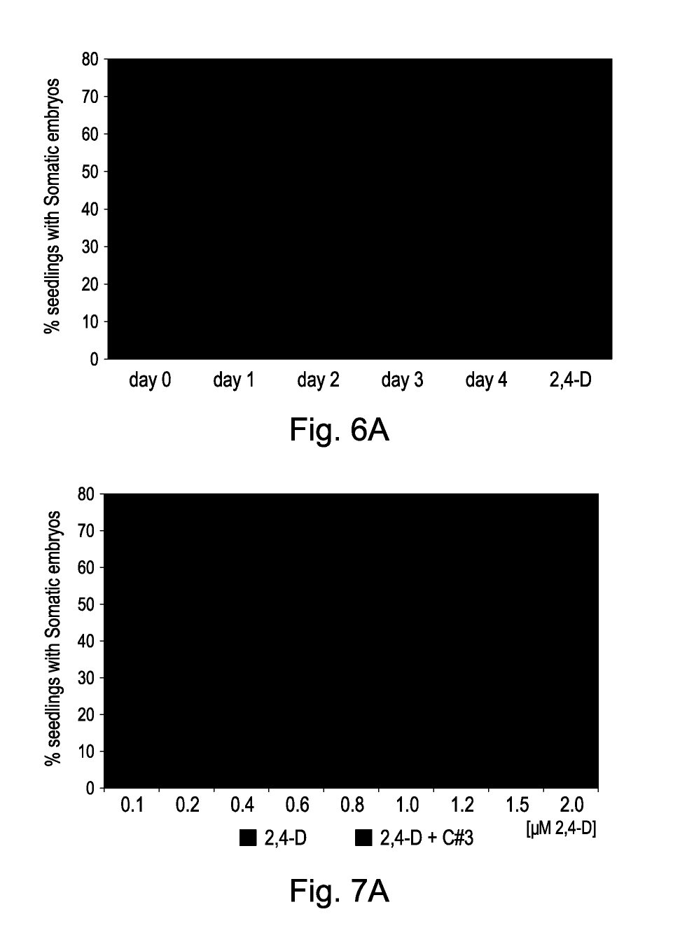

The competence of an explant for in vitro regeneration is determined for a large part by its developmental state. The developmental competence of germinating seedlings to respond to C#3 was examined by adding C#3 at different time points after the start of culture. Seeds were stratified at 4° C. in media containing 1 μM 2,4-D. 25 μM C#3 or the equivalent volume of DMSO (control) was added to the media at different time points after the cultures were transferred to 25° C. The number of seedlings with somatic embryos was determined after two weeks of culture. As shown in FIG. 6 germinating seeds were most responsive to C#3 when C#3 was added within the first two days of culture. Addition of C#3 after two days of culture lead to a decrease in the ability of C#3 to enhance somatic embryogenesis, which reached control levels when added after four days of culture.

### EXAMPLE 4

### 4-chloro-N-methyl-N-(2-methylphenyl)benzenesulfonamide (Compound C#3) Enhances the Auxin Response

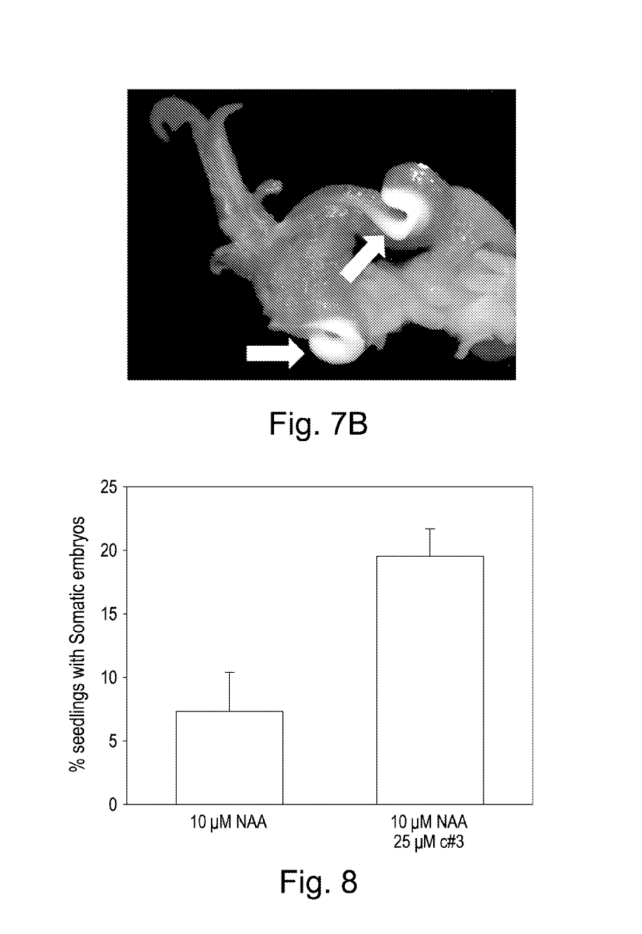

2,4-D is widely used to induce somatic embryogenesis from plant explant. In Arabidopsis immature zygotic embryos occasionally form somatic embryos when grown in basal medium, while germinating embryos or other vegetative tissues are unable to form embryos. The ability of C#3 to enhance somatic embryogenesis was found to be dependent on 2,4-D. Seeds were germinated in medium containing 0 to 2 μM 2,4-D, with or without 25 μM C#3, and then assessed for their ability to form somatic embryos. As shown in FIG. 7A, C#3 was not able to induce somatic embryo formation in the absence of 2,4-D, but did enhance somatic embryo formation at all of the tested 2,4-D concentrations, even when no visible somatic embryos were formed in the control (2,4-D only). Compound #3 therefore potentiates the effect of auxin 2,4-D. As shown in the micrograph in FIG. 7B, at the lowest effective concentration of C#3, areas of dense, light green embryogenic tissue (arrows) were formed, rather than histodifferentiated, bipolar somatic embryos.

The ability of C#3 to enhance embryogenesis in the presence of other auxins was tested; namely the synthetic auxin naphthalene-1-acetic acid (NAA). As shown in FIG. 8, 10 μM NAA induced a low frequency of somatic embryogenesis, which was enhanced by treatment with 25 μM C#3. C#3 therefore enhances the developmental responses that are activated by auxin treatment. Compound C#3 is therefore able to potentiate the effect of other auxins.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A compound or a salt or solvate of a compound of the formula 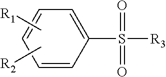
wherein either:
R₁ and R₂ together form an aromatic ring that is fused to the phenyl ring to which R₁ and R₂ are attached, or
R₁ and R₂ are independently selected from H, halogen, halohydrocarbyl, ether, nitro and C₁ to C₅ alkyl functional groups;
R₃ is NR₄R₅,
(i) where R₄ is H or a C₁ to C₅ hydrocarbyl, and where R₅ is H, an amide or —C₆H₂R₆R₇R₈, where R₆, R₇ and R₈ are independently selected from at least one of H, C₁-C₆ hydrocarbyl, halogen, halohydrocarbyl, hydroxyl and an ether functional group, or
(ii) where N, R₄ and R₅ together form a heterocyclic ring,
wherein the compound is not one of the following:

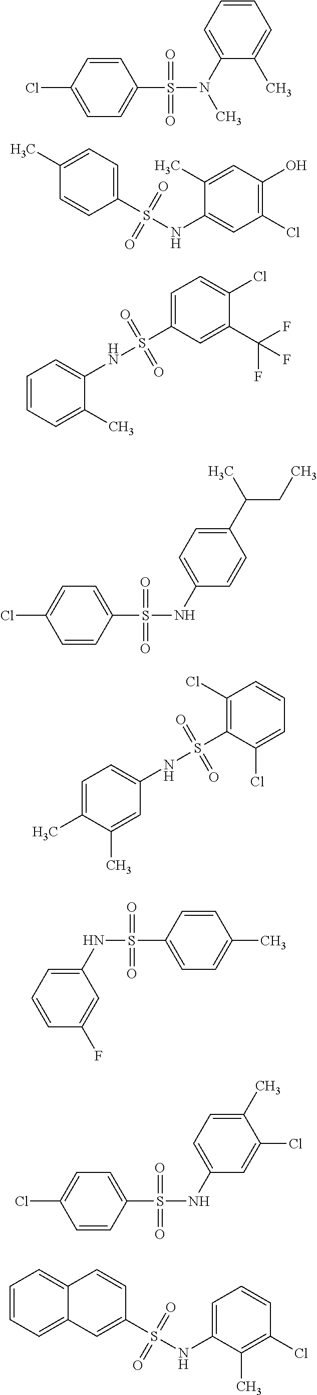

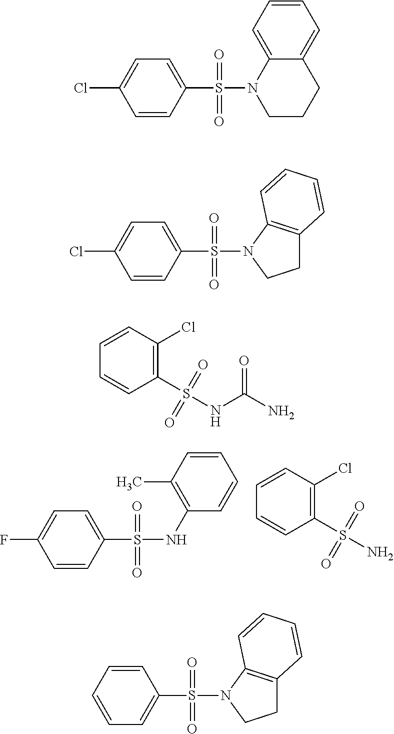

2. A compound as claimed in claim 1, which has the formula: 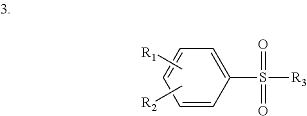
wherein:
R₁ and R₂ together form an aromatic ring that is fused to the phenyl ring to which R₁ and R₂ are attached, or R₁ and R₂ are independently selected from H, halogen, ether, nitro and C₁ to C₅ alkyl functional groups;
R₃ is NR₄R₅, (i) where R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring.

3. A compound as claimed in claim 1 or 2, wherein neither R₄ nor R₅ is H.

4. A compound as claimed in any one of the preceding claims, (i) where R₄ is a C₁ to C₅ hydrocarbyl, and where R₅ is —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring.

5. A compound as claimed in any one of the preceding claims, which is selected from the group consisting of:

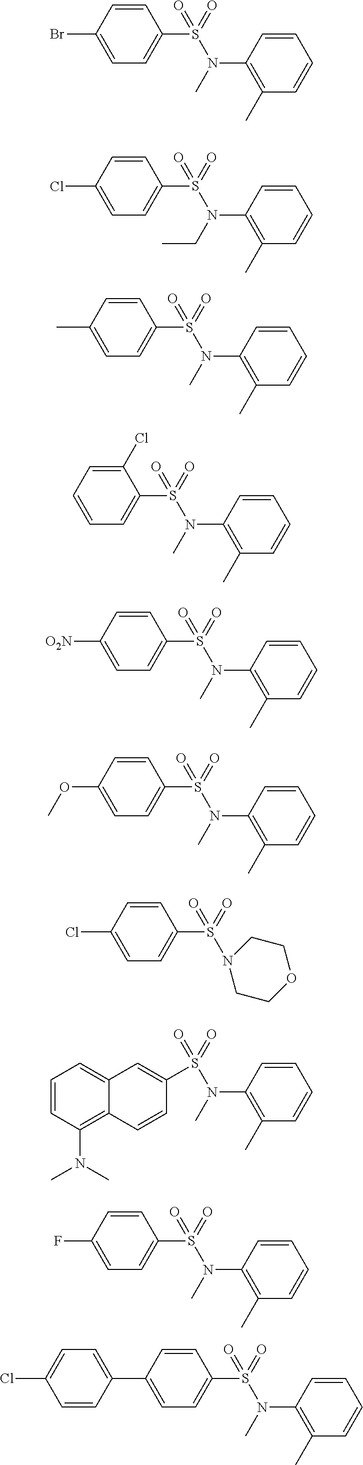

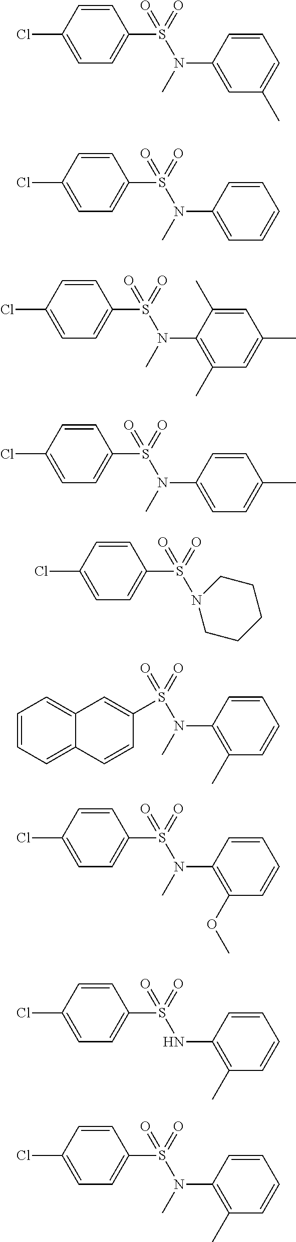

6. A method of potentiating somatic embryogenesis or organogenesis in a plant, comprising exposing plant cells, plant tissue, plant part or plant embryo to an auxin and to one or more compounds of the formula: 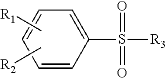
wherein:
R₁ and R₂ together form an aromatic ring that is fused to the phenyl ring to which R₁ and R₂ are attached, or R₁ and R₂ are independently selected from H, halogen, ether, nitro and C₁ to C₅ alkyl functional groups;
R₃ is NR₄R₅, (i) where R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring.
and then culturing the cells, tissue, part or embryo.

7. A method as claimed in claim 6, wherein R₁ and/or R₂ is halogen selected from F, Cl and Br.

8. A method as claimed in claim 6 or claim 7, wherein R₁ and/or R₂ is methyl.

9. A method as claimed in any of claims 6 to 8, wherein one or both of R₁ and R₂ is hydrogen.

10. A method as claimed in any of claims 6 to 8, wherein R₁ and/or R₂ is an ether selected from —OCH₃ and −OC₂H₅.

11. A method as claimed in claim 9, wherein R₁ is hydrogen, and R₂ is a selected from halogen, ether, nitro and C₁ to C₅ alkyl functional groups and is ortho, meta or para, preferably para to the sulfonyl group.

12. A method as claimed in claim 6, wherein where R₁ and R₂ together form a 6-membered aromatic ring that is fused to the phenyl group to which R₁ and R₂ are attached.

13. A method as claimed in claim 6, wherein R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group.

14. A method as claimed in claim 13, wherein where R₄ is H or a C₁ to C₅ alkyl, preferably methyl or ethyl.

15. A method as claimed in claim 13 or claim 14, wherein when at least one of R₆, R₇ and R₈ is a C₁-C₅ alkyl, preferably a methyl group.

16. A method as claimed in claim 13 or claim 14, wherein where at least one of R₆, R₇ and R₈ is an —OCH₃ or —OC₂H₅ group.

17. A method as claimed in any of claims 13 to 16, wherein one or two of R₆, R₇ and R₈ is hydrogen.

18. A method as claimed in claim 17, wherein R₆ is hydrogen and at least one of R₇ and R₈ is selected from a C₁-C₆ hydrocarbyl and an ether functional group.

19. A method as claimed in claim 13, wherein at least one of R₆, R₇ and R₈ is a C₁-C₆ hydrocarbyl or an ether functional group at an ortho, meta or para position.

20. A method as claimed in any of claims 6 to 12, wherein R₃ is NR₄R₅, where N, R₄ and R₅ together form a heterocyclic ring, preferably a 6-membered heterocyclic ring.

21. A method as claimed in claim 20, wherein N, R₄ and R₅ together form a piperidine or morpholine ring.

22. A method as claimed in claim 20 or claim 21, wherein a further aromatic ring is fused to said heterocyclic ring.

23. A method as claimed in claim 22, wherein the aromatic ring is a phenyl ring.

24. A method as claimed in claim 6, wherein R₃ is selected from: 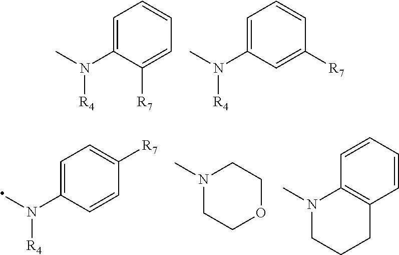
where R₄ is H, CH₃ or C₂H₅; and R₇ is selected from methyl, ethyl, propyl, butyl and OCH₃,
or wherein R₃ is 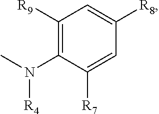
where R₄ is H, CH₃ or C₂H₅; and R₇, R₈ and R₉ are each methyl.

25. A method as claimed in claim 6, wherein 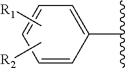
is selected from: 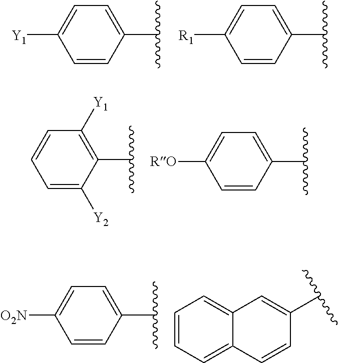
where Y₁ and Y₂ are independently selected from F, Cl, Br, I,
R₁ is methyl or ethyl, and R″ is methyl or ethyl.

26. A method as claimed in any of claims 6 to 25, wherein the plant cells, tissue, part or embryo is, or includes, callus.

27. A method as claimed in any of claims 6 to 26, wherein the plant cells, tissue, part or embryo are exposed to the auxin and the one or more compounds substantially simultaneously.

28. A method as claimed in any of claims 6 to 26, wherein the plant cells, tissue, part or embryo are exposed to the auxin followed by the one or more compounds.

29. A method as claimed in any of claims 6 to 26, wherein the plant cells, tissue, part or embryo are exposed to the one or more compounds followed by the auxin.

30. A method as claimed in any preceding claim, wherein a first period of exposure is followed by a second period of culturing in the absence of either the auxin and/or the one or more compounds.

31. A method as claimed in any preceding claim, wherein the exposing of the plant cells, tissue, part or embryo takes place in a liquid medium.

32. A method as claimed in claim 30 or claim 31, wherein culturing of the cells, tissue, part or embryo takes place on a solid medium.

33. A method as claimed in any preceding claim, wherein the auxin is selected from one or more of: indole-3-acetic acid (IAA), indole-3-butyric acid (IBA), 4-chloroindole-3-acetic acid (4-Cl-IAA), 2-phenylacetic acid (PAA), 2.4-dichlorophenoxyacetic acid (2,4-D), α-napthalene acetic acid (α-NAA), synthetic auxins, e.g., 2-methoxy-3,6-dichlorobenzoic acid (dicamba), 4-amino-3,5,6-trichloropicolinic acid (tordon or picloram).

34. A method as claimed in claim 33, wherein the auxin is 2,4 dichlorophenoxyacetic acid (2,4-D).

35. A method of generating plantlet or plant, comprising producing a somatic embryo as claimed in a method of any of claims 6 to 34 and then regenerating the plantlet or plant from the embryo.

36. A composition for potentiating somatic embryogenesis or organogenesis in plants comprising an auxin and one or more compounds of the formula: 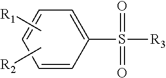
wherein:
R₁ and R₂ together form an aromatic ring that is fused to the phenyl ring to which R₁ and R₂ are attached, or R₁ and R₂ are independently selected from H, halogen, ether, nitro and C₁ to C₅ alkyl functional groups;
R₃ is NR₄R₅, (i) where R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring.

37. Compounds of the formula: 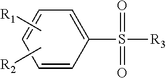
wherein:
R₁ and R₂ together form an aromatic ring that is fused to the phenyl ring to which R₁ and R₂ are attached, or R₁ and R₂ are independently selected from H, halogen, ether, nitro and C₁ to C₅ alkyl functional groups;
R₃ is NR₄R₅, (i) where R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring, for use in the potentiation of somatic embryogenesis or organogenesis in a plant cell, tissue, part or embryo.

38. Compounds of the structure: 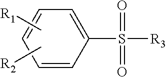
wherein:
R₁ and R₂ together form an aromatic ring that is fused to the phenyl ring to which R₁ and R₂ are attached, or R₁ and R₂ are independently selected from H, halogen, ether, nitro and C₁ to C₅ alkyl functional groups;
R₃ is NR₄R₅, (i) where R₄ is H or a C₁ to C₅ hydrocarbyl, and where R₅ is H or —C₆H₂R₆R₇R₈, where R₆, R₇ and R₈ are independently selected from at least one of H, C₁-C₆ hydrocarbyl and an ether functional group, or (ii) where N, R₄ and R₅ together form a heterocyclic ring;
for use simultaneously, sequentially or separately with an auxin in the potentiation of somatic embryogenesis or organogenesis in a plant cell, tissue, part or embryo.

39. Compounds as claimed in claim 37 or claim 38, wherein the compound has a structure as claimed in any of claims 7 to 20.

40. A solid or liquid plant culture medium comprising one or more compounds as set forth in any of claims 6 to 20.

41. A medium as claimed in claim 40, further comprising an auxin.

42. A kit for potentiating somatic embryogenesis or organogenesis in plants, comprising a first container containing a substance which is or comprises one or more compounds as defined in any of claims 6 to 20.
